(19) United States
(12) Patent Application Publication
(10) Pub. No.: US 2025/0306031 A1
(43) Pub. Date: Oct. 2, 2025

(54) COMPOSITIONS AND METHODS OF TREATING SEPSIS IN PATIENTS USING ANTI-LIGHT ANTIBODIES

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Hakon HAKONARSON, Malvern, PA (US); Huiqi QU, Drexel Hill, PA (US); Scott WEISS, Glen Mills, PA (US); Patrick SLEIMAN, Philadelphia, PA (US); Nuala MEYER, Wynnewood, PA (US)

(73) Assignees: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(21) Appl. No.: 18/557,834

(22) PCT Filed: May 2, 2022
(86) PCT No.: PCT/US2022/027325

§ 371 Date: Oct. 27, 2023

Related U.S. Application Data
(60) Provisional application No. 63/182,531, filed on Apr. 30, 2021.

(51) Int. Cl. G01N 33/68

(52) U.S. Cl. CPC G01N 33/6863; G01N 2333/525; G01N 2333/54; G01N 2800/26

(57) ABSTRACT

The present disclosure relates to methods of treating sepsis or septic conditions in patients who have heightened LIGHT, IL-18 levels or biomarkers listed herein which can be treated with molecules that inhibit biomarker activity.

### CROSS-REFERENCE TO RELATED APPLICATIONS

This application claims priority to U.S. Provisional Application No. 63/182,531 filed Apr. 30, 2021, which is incorporated herein by reference as though set forth in full.

### FIELD OF THE INVENTION

The present disclosure relates the field of inflammatory disorders, more particularly sepsis associated with microbial and viral infections. More specifically, the invention discloses compositions and methods for treating inflammatory disorders having elevated expression of LIGHT and other biomarkers.

### BACKGROUND OF THE INVENTION

Several publications and patent documents are cited throughout the specification in order to describe the state of the art to which this invention pertains. Each of these citations is incorporated by reference herein as though set forth in full.

The cytokine LIGHT (CD258), also known as tumor necrosis factor superfamily member 14 (TNFSF14), is a secreted protein of the TNF superfamily, recognized by the herpesvirus entry mediator (HVEM), the lymphotoxin B receptor (LTBR), and by decoy receptor, DcR31. LIGHT exhibits inducible expression and competes with herpes virus glycoprotein D for binding to HVEM on T lymphocytes². LIGHT is a ligand for TNFRSF14, which is a member of the tumor necrosis factor receptor superfamily, also known as HVEM ligand (HVEML). This protein functions as a costimulatory factor for the activation of lymphoid cells aimed at opposing infection by herpesvirus². It additionally stimulates the proliferation of T cells, and triggers apoptosis of various tumor cells³.

Inflammatory reactions and immune responses are known to be dysregulated and compromised in patients with septicemia, many of whom sustain serious organ injuries⁶. In this regard, acute respiratory distress syndrome (ARDS) and acute kidney injury (AKI) are major complications of severe infections^{7,8}, while the mechanisms of immune dysfunction causing these complications remains unknown. Sepsis typically manifests as an uncontrolled systemic inflammatory process, involving the microvasculature of multiple organ systems, causing disseminated intravascular coagulation (DIC)⁹, with ARDS as the pulmonary manifestation, and AKI as the kidney manifestation^{10,11}. Liver, heart, and brain failures may also occur in the most severe cases.

LIGHT, has recently come into the spotlight as a potent pro-inflammatory mediator and has been suggested as an important therapeutic target for immune regulation due to its central role in the function of activated T cells¹²⁻¹⁴. Previous study showed that soluble LIGHT induces proinflammatory changes in endothelial cells under systemic inflammatory activation¹⁵. LIGHT's role in initiating inflammation and tissue fibrosis implies its involvement in COVID related ARDS. Indeed, several studies have demonstrated elevated LIGHT levels in serum of COVID ARDS patients¹⁶⁻¹⁸. A follow up clinical trial using LIGHT neutralizing mAb was successful in reducing lung injury, ventilator time in ICU, hospital stay and mortality¹⁹. A recent study also suggested an important role of LIGHT/HVEM expression in experimental lung injury in mice²⁰.

### SUMMARY OF THE INVENTION

In accordance with the present invention, a method is provided for the treatment of sepsis in patients having an elevated APACHE III score two (2) standard deviations above the mean observed in healthy control subjects. An exemplary method comprises determining the APACHE III score followed by administration of an effective amount of a sepsis biomarker inhibitor to the patient. APACHE III scores in the range of at least 200 to 299 indicate the need for treatment with a LIGHT antagonist. In certain embodiments, a score of 210, 225, 250, 275, to 300 is calculated. Also provided is a method of treating sepsis in a patient in need thereof, comprising: a) determining whether the patient harbors an elevated level of at least one sepsis biomarker, and b) administering an effective amount of a sepsis biomarker inhibitor. In certain embodiments, the sepsis biomarker is LIGHT. In preferred embodiments, the sepsis biomarker inhibitor is an anti-LIGHT antibody.

Also provided is a method of treating sepsis in a patient in need thereof, comprising: a) determining whether the patient harbors: i) an elevated level of at least two sepsis biomarkers selected from LIGHT, TIMP-1, TNFR2, VCAM-1, PAI-1, IL-18, IL-18BP, IL-6, vWF, IL-8, FRTN, IL-1RA, MMP-3, IL-10, Eotaxin-1, MIP-1β, and IL-1β when compared to the mean observed in control subjects; and/or ii) a lower level of at least two sepsis biomarkers selected from complement C3, Factor VII, Vitamin D-Binding Protein (VDBP), Thyroxine-Binding Globulin (TBG), Serum Amyloid P-Component (SAP), Fibrinogen, and T-Cell-Specific Protein RANTES (RANTES) when compared to the mean observed in control subjects; and b) administering an effective amount of a sepsis biomarker inhibitor.

Also provided is a method of treating Acute Respiratory Distress Syndrome (ARDS) not caused by Covid infection, in a patient in need thereof, comprising: a) determining whether the patient harbors: i) an elevated of at least two sepsis biomarker selected from IL-10, IL-6, IL-1Ra, IL-8, TIMP-1, and PAI-1 when compared to the mean observed in control subjects; and/or ii) a lower level fibronectin when compared to the mean observed in control subjects; and b) administering an effective amount of a sepsis biomarker inhibitor.

Also provided is a method of treating Acute Kidney Injury (AKI) in a patient in need thereof, comprising; a) determining whether the patient harbors an elevated of at least two sepsis biomarker selected from B2M, Stem Cell Factor (SCF), TNFR2, VCAM-1, TIMP-1, Myoglobin, MMP-3, PAI-1, IL-18, and IL18BP; and b) administering an effective amount of a sepsis biomarker inhibitor.

### BRIEF DESCRIPTION OF THE DRAWINGS

FIG. 1. Graphical representation of increased Ln (LIGHT) levels in bacterial and viral sepsis.

FIG. 2A-2B. Table displaying correlation of elevated LIGHT and Ln(IL-18) with biomarkers of organ failure.

FIG. 3. Table displaying Sixty-One Inflammation biomarkers investigated in the Examples.

### DETAILED DESCRIPTION OF THE INVENTION

In accordance with the present invention, the inventors have recognized that inhibitors of sepsis biomarkers such as LIGHT and other biomarkers, for example anti-LIGHT antibodies or small molecule inhibitors, may be particularly useful in treating sepsis in subjects who have elevated levels of LIGHT or IL-18.

To investigate the potential role of LIGHT, IL18 and other inflammation-related cytokine mediators in bacterial and viral-induced sepsis, as well as their roles in the major complications of sepsis, including but not limited to ARDS and AKI that typically result from more severe infections, plasma levels of LIGHT and IL-18 were measured together with 59 inflammation or inflammasome biomarkers in 280 patients with sepsis. Of those, 189 had either culture proven or presumed bacterial sepsis and 91 had culture-proven or presumed viral sepsis that resulted in hospitalization and admission to the intensive care unit (ICU).

The significant roles of LIGHT and other biomarkers in the severity of sepsis are highlighted herein. For the first time, we demonstrate a key damaging role of LIGHT in patients with sepsis complicated by ARDS or multi-organ failures.

The following definitions are provided to facilitate an understanding of the invention. They are not intended to limit the invention in any way.

### Definitions

For purposes of the present invention, “a” or “an” entity refers to one or more of that entity; for example, “an antibody” refers to one or more antibodies or at least one antibody. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds.

According to the present invention, an “isolated,” or “biologically pure” molecule is a compound that has been removed from its natural milieu. As such, the terms “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “sepsis” refers an extreme inflammatory response to infection. This inflammation occurs due to inflammatory cytokines being produced throughout the body. The infection can be due to bacteria in the bloodstream (septicemia) or a virus, but sepsis can also be produced by an infection that is present only in one part of the body, such as the lungs in pneumonia. The inflammation in sepsis can produce blood clots and leaking blood vessels. Without proper treatment, this can lead to damage in vital organs and death. Sepsis can progress to septic shock when the patient's blood pressure drops and the patient's body starts to shut down. The patient's lungs, liver and kidneys can fail.

The term “septicemia” refers to a bacterial infection that spread into the bloodstream.

The phrase “acute respiratory distress syndrome” or “ARDS” refers to a condition in which fluid collects in the lungs' air sacs, depriving organs of oxygen. ARDS can occur in those who are critically ill or who have significant injuries. ARDS is often fatal. ARDS is often caused by sepsis. Symptoms of ARDS include severe shortness of breath and inability to breathe without support.

The phrase “acute hypoxemic respiratory failure” or “AHRF” refers to severe arterial hypoxemia caused by intrapulmonary shunting of blood resulting from airspace filling or collapse or by intracardiac shunting of blood from the right-to left-sided circulation. AHRF can be caused by sepsis.

The phrase “acute kidney injury” or “AKI” refers to an abrupt decrease in kidney function, resulting in the retention of urea and other nitrogenous waste products and in the dysregulation of extracellular volume and electrolytes.

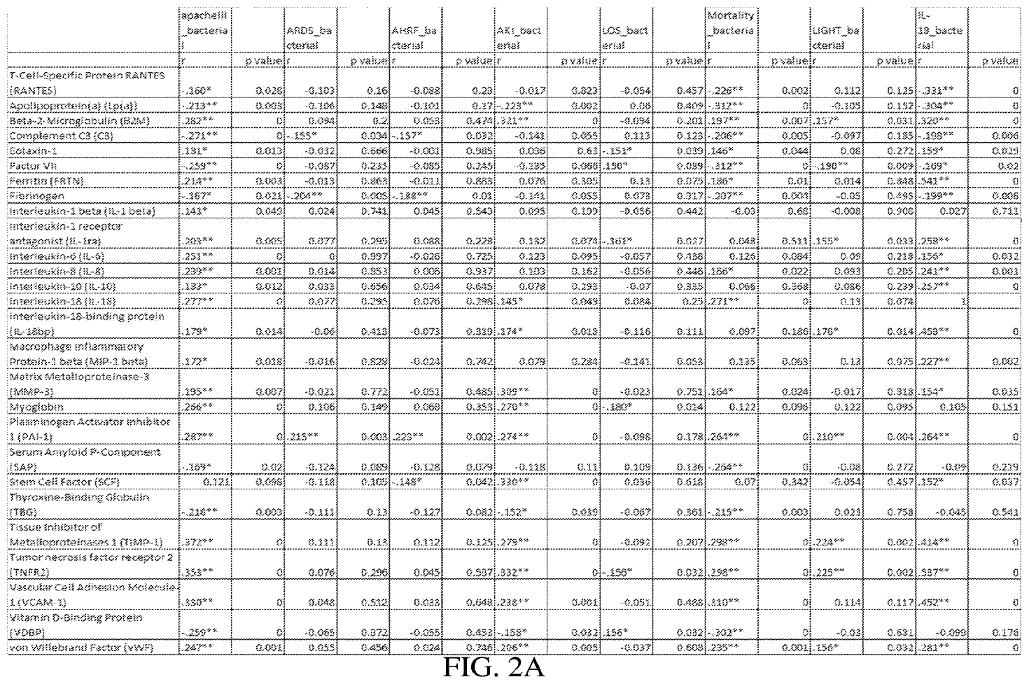

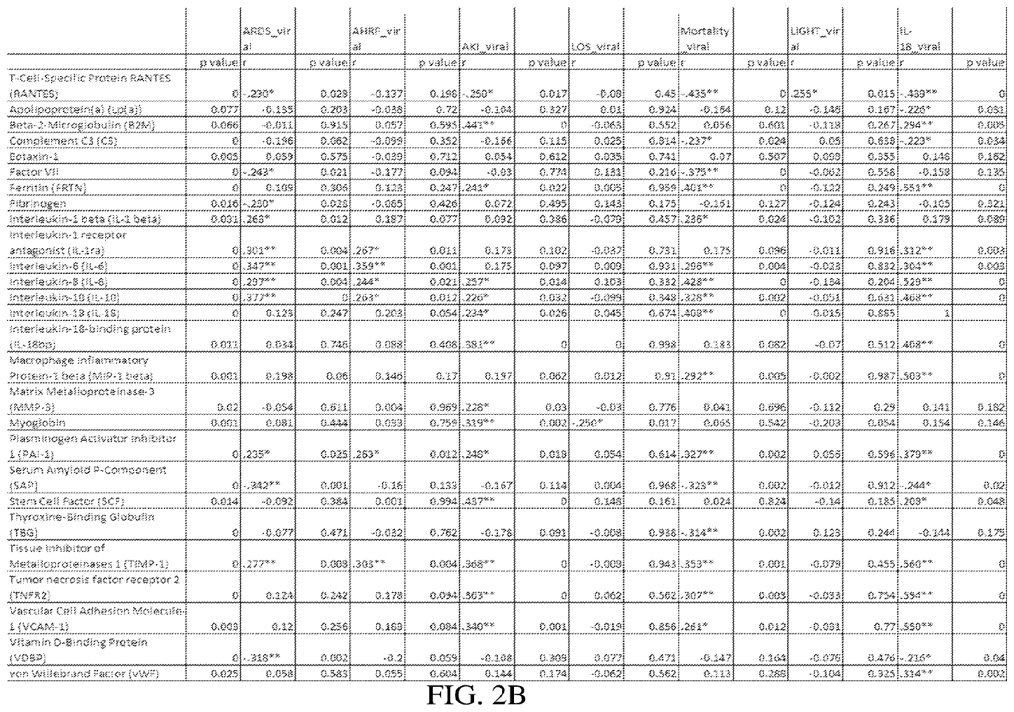

The phrase “related marker” as used herein with regard to a biomarker such as one of the biomarkers (i.e., for example, a sepsis biomarker) described herein (See FIG. 2). A related marker may also refer to one or more fragments, variants, etc., of a particular marker or its biosynthetic parent that may be detected as a surrogate for the marker itself or as independent biomarkers. The term also refers to one or more polypeptides present in a biological sample that are derived from the biomarker precursor complexed to additional species, such as binding proteins, receptors, heparin, lipids, sugars, etc.

The phrase “sepsis biomarker” or “septicemia biomarker” as used herein, refers to any biological compound related to the progressive development of sepsis. For example, a septicemia biomarker may comprise, without limitation, LIGHT, IL-18, or the biomarkers present in FIG. 2. A sepsis biomarker is considered elevated when it is >2 standard deviations above the mean in reference controls.

The phrase “The Acute Physiology and Chronic Health Evaluation III score” or “APACHE III score” refers to an index typically used to measure the severity of disease of ICU patients. This point score, calculated as a sum of physiologic variables, age, and chronic health points for each ICU patient, is used not only to evaluate severity of disease but also as a prognostic predictor. While physiologic variables directly reflect the severity of disease, age and chronic health points are background factors contributing to disease severity. Besides the APACHE III score, factors such as gender, past medical history, and infection have been reported to influence the prognosis of ICU patients. The APACHE III scoring was as described by Knaus et al., as previously reported²⁸. A high APACHE III score is defined as a score >2 standard deviations (SD) above mean score in reference controls. A high APACHE III score is indicative of elevated sepsis biomarker levels.

The term “sepsis biomarker inhibitor” or “septicemia biomarker inhibitor” refers to a molecule that inhibits the function of a septicemia biomarker. Septicemia biomarker inhibitors may include small molecules or biologics, and may include antagonist antibodies that bind to sepsis biomarkers such LIGHT, as well as proteins that act as traps for those ligands. Sepsis biomarker inhibitors are well known by those skilled in the art. For example, inhibitors of LIGHT include, without limitation, the anti-LIGHT antibodies described herein, antirheumatic drugs, arsenous acid, bisphenol A, cyclosporin A, diarsenic trioxide, isoflurane, N-Nitrosopyrrolidine, p-menthan-3-ol, paracetamol, pentobarbital, perfluorononanoic acid, perfluoroundecanoic acid, silicon dioxide, vinclozolin, methotrexate, Baminercept, and SAR252067 (See also Gene: Tnfsf14 (TNF superfamily member 14) homosapiens, available on the world wide web at: rgd.mcw.edu/rgdweb/report/gene/main.html?id=1352930, which is incorporated herein by reference).

Inhibitors of IL-18 include, without limitation, 1,2-dichloroethane, 1,2-dimethylhydrazine, 17alpha-ethynylestradiol, 2,3,7,8-tetrachlorodibenzodioxine, and 4,4′-diaminodiphenylmethane. (See also, Gene: IL18 (interleukin 18) homosapiens, available on the world wide web at: rgd.mcw.edu/rgdweb/report/gene/main.html?id=730894, which is incorporated herein by reference).

Inhibitors of IL-18BP include, without limitation, endosulfan, leflunomide, tetrachloromethane, thioacetamide, and oxycodone. (See also, Gene: IL18BP (interleukin 18 binding protein) homosapiens, available on the world wide web at: rgd.mcw.edu/rgdweb/report/gene/main.html?id=1348622, which is incorporated herein by reference).

Inhibitors of IL-6 include, without limitation, 2,3,7,8-Tetrachlorodibenzofuran, 2-acetamidofluorene, 2-arachidonoylglycerol, 4-hydroxynon-2-enal, and acetylsalicylic acid. (See also, Gene: IL6 (interleukin 6) homosapiens, available on the world wide web at: rgd.mcw.edu/rgdweb/report/gene/main.html?id=1352582, which is incorporated herein by reference).

Inhibitors of IL-10 include, without limitation, cyhalothrin, dextran sulfate, dextromethorphan, diazinon, and disodium selenite. (See also, Gene: IL10 (interleukin 10) homosapiens, available on the world wide web at: rgd.mcw.edu/rgdweb/report/gene/main.html?id=735591, which is incorporated herein by reference).

Inhibitors of IL-1β include, without limitation, acrylamide, betalain, carvedilol, methotrexate, and lansoprazole. (See also, Gene: IL1B (interleukin 1 beta) homosapiens, available on the world wide web at: rgd.mcw.edu/rgdweb/report/gene/main.html?id=730981, which is incorporated herein by reference).

Inhibitors of TIMP-1 include, without limitation, cucurbitacin E, dexamethasone, doxorubicin, gentamycin, and ketoconazole. (See also, Gene: TIMP-1 (TIMP metallopeptidase inhibitor 1) homosapiens, available on the world wide web at: rgd.mcw.edu/rgdweb/report/gene/main.html?id=1347215, which is incorporated herein by reference).

Inhibitors of VCAM-1 include, without limitation, amlodipine, benazepril, biochanin A, chloroprene, and clobetasol. (See also, Gene: VCAM1 (vascular cell adhesion molecule 1) homosapiens, available on the world wide web at: rgd.mcw.edu/rgdweb/report/gene/main.html?id=730988, which is incorporated herein by reference).

Inhibitors of vWF include, without limitation, bisphenol A, dibutyl phthalate, enalapril, indometacin, and trichloroethene (See also, Gene: VWF (von Willebrand factor) homosapiens, available on the world wide web at: rgd.mcw.edu/rgdweb/report/gene/main.html?id=1347936, which is incorporated herein by reference).

Inhibitors of MMP-3 include, without limitation, 1,2-dichloroethane, 17beta-estradiol, avobenzone, diethylstilbestrol, and enalapril. (See also, Gene: MMP3 (matrix metallopeptidase 3) homosapiens, available on the world wide web at: rgd.mcw.edu/rgdweb/report/gene/main.html?id=1345848, which is incorporated herein by reference).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO: or compound. For example, when used in reference to an amino acid sequence, the phrase includes the sequence per se and molecular modifications that would not affect the functional and novel characteristics of the sequence. Similarly, the phrase refers to compounds with modifications that do not affect the functional and novel characteristics of the parent compound. Methods can also consist essentially of a recited series of steps.

“Target nucleic acid” as used herein refers to a previously defined region of a nucleic acid present in a complex nucleic acid mixture wherein the defined wild-type region contains at least one known nucleotide variation that may or may not be associated with ARDS/AHRF/AKI. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process includes the construction of a cDNA library from mRNA and isolation of distinct cDNA clones and yields an approximately 10⁶ fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule. Levels of complementarity between selectively hybridizing nucleic acids can vary but is typically greater than 80% and is preferably between 90-95%.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”) with enough sequence specificity to distinguish the target sequence over non-target sequences. In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence that hybridizes to any AID specific marker nucleic acid, but does not hybridize to other nucleotides. Also, polynucleotide that “specifically hybridizes” may hybridize only to a single AID-specific marker shown in the Tables contained herein. Appropriate conditions enabling specific hybridization of single stranded nucleic acid molecules of varying complementarity are well known to those of skill in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

$${T}_{m} = 81.5 ° C . + 16.6 Log [ Na + ] + 0.41 ( \% G + C ) - 0.63 ( \% formamide ) - 600 / \# bp in duplex$$

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6× SSC, 5× Denhardt's solution, 0.5% SDS and 100 μg/ml denatured salmon sperm DNA at 42° C., and washed in 2× SSC and 0.5% SDS at 55° C. for 15 minutes. A high stringency hybridization is defined as hybridization in 6× SSC, 5× Denhardt's solution, 0.5% SDS and 100 μg/ml denatured salmon sperm DNA at 42° C., and washed in 1× SSC and 0.5% SDS at 65° C. for 15 minutes. A very high stringency hybridization is defined as hybridization in 6× SSC, 5× Denhardt's solution, 0.5% SDS and 100 μg/ml denatured salmon sperm DNA at 42° C., and washed in 0.1× SSC and 0.5% SDS at 65° C. for 15 minutes.

The term “oligonucleotide,” as used herein is defined as a nucleic acid molecule comprised of two or more ribo-or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) that have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long. Typically, one or both members of a specific binding pair will comprise a non-naturally occurring detectable label.

“Sample” or “patient sample” or “biological sample” generally refers to a sample which may be tested for a particular molecule, such as a sepsis biomarker, such as a biomarker shown in the FIG. 2. Samples may include but are not limited to cells, body fluids, including blood, serum, plasma, urine, saliva, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the biomarkers described herein or their encoded proteins. Agents are evaluated for potential biological activity by inclusion in screening assays described hereinbelow.

The term “reagent” as used herein, refers to any substance employed to produce a chemical reaction so as to detect, measure, produce, etc., other substances.

A “patient” or “subject” as referred to herein may be either an adult (18 or older) or a pediatric subject (under 18). These two terms are generally used interchangeably herein.

“Treatment,” as used herein, covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process.

The term “diagnosis” as used herein, refers to methods by which trained medical personnel can estimate and/or determine the probability (i.e., for example, a likelihood) of whether or not a patient is suffering from a given disease or condition. In certain embodiments of the present invention, “diagnosis” includes correlating the results of an assay (i.e., for example, an immunoassay) for a sepsis biomarker of the present invention, optionally together with other clinical indicia, to determine the occurrence or nonoccurrence of sepsis for a subject or patient from which a sample was obtained and assayed. That such a diagnosis is “determined” is not meant to imply that the diagnosis is 100% accurate. Thus, for example, a measured biomarker level below a predetermined diagnostic threshold may indicate a greater likelihood of the occurrence of a disease in the subject relative to a measured biomarker level above the predetermined diagnostic threshold may indicate a lesser likelihood of the occurrence of the same disease. In certain embodiments, no assay is performed and other clinical indicia, such as APACHE-III scores, are used to determine the likelihood of sepsis for a patient.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

### Identification of Biomarker Levels

In some embodiments, a subject, or a biological sample from a subject, is assayed to determine the presence or absence of increased levels of inflammatory biomarkers. In some embodiments, the biomarker is associated with altered activity of one or more genes shown in FIG. 2, particularly LIGHT. In some embodiments, the increased biomarker level is associated with increased septic activities such as serious organ injuries. In other embodiments, reduction of biomarker expression is correlated with sepsis.

In some embodiments, information regarding whether a patient has increased levels of inflammatory biomarkers is analyzed, and treatment is initiated based upon this information.

In some embodiments, the biomarker is associated with increased levels or activity of LIGHT. Thus, in some embodiments, the methods encompass determining whether a subject has a biomarker that is associated with increased levels or activity of LIGHT.

FIG. 2 provides a list of biomarkers associated with sepsis that are enriched in sepsis cases compared to controls. In some embodiments, the subject may harbor elevated levels of one or more of those biomarkers.

In some embodiments, assays may be conducted with a variety of samples such as blood, urine, serum, and gastric lavage bodily fluid samples and cell samples such as white blood cells or mononuclear cells.

### Treatment of Sepsis in Patients Having Increased Biomarker Levels

This disclosure encompasses methods of treating sepsis or septicemia with a sepsis biomarker inhibitor such as a LIGHT antagonist. In some embodiments, the subject is treated with an anti-LIGHT antibody.

In some embodiments, the methods encompass determining whether the patient has a heightened levels of at least one sepsis biomarker and, if heightened levels are detected, treating the patient with a sepsis biomarker inhibitor.

In some embodiments, the inhibitor is a small molecule, while in other embodiments the inhibitor is a biologic, such as an antibody, a ligand trap, an aptamer, or a nucleic acid such as a small inhibiting RNA (siRNA) or antisense nucleic acid. In some embodiments, the inhibitor is an antibody, such as an antagonist antibody of LIGHT. In some embodiments, the biomarker inhibitor is specific to one sepsis biomarker.

Suitable anti-LIGHT antibodies for the present treatment methods include those described, for example, in WO 2008/027338, US20130315913, US20130323240, and WO 2015/107331, which are incorporated herein by reference in their entirety. In some embodiments, the anti-LIGHT antibody inhibits a biological function of LIGHT, such as binding to one of its ligands, such as HVEM or LTβR.

Inhibitors may be administered systemically in parenteral, oral solid and liquid formulations, ophthalmic, suppository, aerosol, topical or other similar formulations. In addition to the appropriate active ingredient, such as a sepsis biomarker inhibitor, an appropriate pharmaceutical composition may contain pharmaceutically-acceptable carriers and other ingredients known to enhance and facilitate drug administration. Thus, such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the active ingredient to a patient according to the methods of the invention. The use of nanoparticles to deliver siRNAs or expression vectors, as well as cell membrane permeable peptide carriers that can be used are described in Crombez et al., Biochemical Society Transactions v35:p44 (2007).

### Anti-LIGHT Antibodies

In some embodiments, the patient is treated with an anti-LIGHT antibody. The anti-LIGHT antibody may comprise the CDR sequences of the E1, E13, E63, F19, or F23 antibodies, which are provided in WO 2008/027338 and U.S. Pat. Nos. 8,058,402 B2, 8,461,307 B2, and 8,974,787 B2, each of which is incorporated herein by reference.

In some embodiments, the anti-LIGHT antibody may comprise the CDR sequences of the antibodies, which are described in US2013/0323240 and U.S. Pat. No. 8,524,869 B2, which are incorporated herein by reference.

In some embodiments, the anti-LIGHT antibody may comprise the CDR sequences of the antibodies, which are described in U.S. Pat. No. 10,407,725, which is incorporated herein by reference.

In some embodiments, the anti-LIGHT antibody may comprise a heavy chain and a light chain together comprising one of the following sets of CDR-H1, CDR-H2, CDR-H3, CDR-L1, CDR-L2, and CDR-L3 sequences described in the sequence listing from US2013/0323240: SEQ ID NOs: 18, 19, 20 and SEQ ID NOs: 38, 41, 42 of US2013/0323240; SEQ ID NOs: 18, 19, 21 and SEQ ID NOs: 39, 41, 42 of US2013/0323240; SEQ ID NOs: 18, 19, 22 and SEQ ID NOs: 40, 41, 42 of US2013/0323240; SEQ ID NOs: 23, 24, 25 and SEQ ID NOs: 43, 44, 45 of US2013/0323240; SEQ ID NOs: 26, 27, 28 and SEQ ID NOs: 46, 47, 48 of US2013/0323240; SEQ ID NOs: 29, 30, 31 and SEQ ID NOs: 49, 50, 51 of US2013/0323240; SEQ ID NOs: 32, 33, 34 and SEQ ID NOs: 52, 53, 54 of US2013/0323240; and SEQ ID NOs: 35, 36, 37 and SEQ ID NOs: 55, 50, 51 of US2013/0323240.

In some embodiments, the anti-LIGHT antibody comprises the CDR sequences of the 18E04, 98C07, 1C02, 1C06, 13H04, 31A10, 98C07, 42A02, 29C02, 14B09, 117C06, 114F05, and 62C01 antibodies described in WO 2015/107331, which is also incorporated by reference herein.

### Kits

In some embodiments, the present invention also contemplates devices and kits for performing the methods described herein. Suitable kits comprise reagents sufficient for performing an assay for at least one of the described biomarkers, together with instructions for performing the described threshold comparisons.

In certain embodiments, the aforementioned products can be incorporated into a kit which may contain a sepsis biomarker specific marker polynucleotide or one or more such markers immobilized on a solid support or a Gene Chip. The kit may also comprise an oligonucleotide, a polypeptide, a peptide, an antibody, one or more non-naturally occurring detectable labels, marker, or reporter, a pharmaceutically acceptable carrier, a physiologically acceptable carrier, instructions for use, a container, a vessel for administration, an assay substrate, or any combination thereof.

In certain embodiments, reagents for performing such assays are provided in an assay device, and such assay devices may be included in such a kit. Preferred reagents can comprise one or more solid phase antibodies, the solid phase antibody comprising antibody that detects the intended biomarker target(s) bound to a solid support. In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter.

In some embodiments, the present invention provides kits for the analysis of the described biomarkers. The kit comprises reagents for the analysis of at least one test sample which comprise at least one antibody that a sepsis biomarker. The kit can also include devices and instructions for performing one or more of the diagnostic and/or prognostic correlations described herein. Preferred kits may comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies that bind a kidney injury marker. Most preferably each of the antibodies are monoclonal antibodies. The instructions for use of the kit and performing the correlations can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

The following examples are provided to illustrate certain embodiments of the invention. They are not intended to limit the invention in any way.

### EXAMPLES

A total of 280 cases diagnosed with sepsis, including 91 cases with sepsis triggered by viral infections, were investigated. Serum LIGHT, IL-18, and 59 other biomarkers (cytokines, chemokines and acute-phase reactants) were measured and correlated with symptom severity.

### Subjects

A total of 280 sepsis cases, including 189 (67.5%) cases diagnosed with bacterial sepsis (either culture proven or presumed based on clinical presentation) and 91 (32.5%) cases diagnosed with viral sepsis (either culture/PCR proven or presumed based on clinical presentation), were investigated in this study (Table 1), and were compared with 22 random population-based controls.

TABLE 1
Clinical characteristics of the research subjects

| Clinical | Bacterial | Viral |
|---|---|---|
| Information | Sepsis | Sepsis |
| Age (median, 1^{st} | 61.5 (52.1, 71.4) | 61 (52.5, 69) |
| and 3^{rd} quartiles) | | |
| Sex | 111(58.7%) males; | 51(56.0%) males; |
| | 78(41.3%) females | 40(44.0%) females |
| BMI (x̅ ± s) | 27.05 ± 7.70 | 27.59 ± 7.37 |
| Race | White: 121; Black: | White: 55; Black: |
| | 57; Asian: 8; | 31; Asian: 2; |
| | Native American: 1; | Other: 3 |
| | Other: 2 | |
| ARDS | Yes: 65(34.4%); | Yes: 36(39.6%); |
| | No: 124(65.6%) | No: 55(60.4%) |
| AHRF | Yes: 71(37.8%); | Yes: 50(55.6%); |
| | No: 117(62.2%) | No: 40(44.4%) |
| AKI | Yes: 114(61.6%); | Yes: 49(53.8%); |
| | No: 71(38.4%) | No: 42(46.2%) |
| APACHE III | 101.6 ± 36.9 | 90.8 ± 39.4 |
| LOS* | 19.38 ± 20.78 | 17.70 ± 19.98 |
| Mortality | Yes: 77(40.7%); | Yes: 33(36.3%); |
| | No: 112(59.3%) | No: 58(63.7%) |

*length of ICU/hospital stay

The diagnosis of ARDS was based on the Berlin definition²⁵. The diagnosis of acute hypoxic respiratory failure (AHRF) was based on previously reported criteria defining hypoxemia using oxygen indices²⁶. The diagnosis of AKI was made in patients without preexisting chronic kidney disease, based on serum creatinine and urine output using the Kidney Disease Improving Global Outcomes (KDIGO) guidelines²⁷. The APACHE III scoring was as described by Knaus et al., as previously reported²⁸.

Among the patients with culture or PCR proven viral sepsis, 56 (61.5%) had influenza; 10 (11.0%) had respiratory syncytial virus (RSV); 7 (7.7%) had Human metapneumovirus (HMPV); 4 (4.4%) had adenoviruses; 4 (4.4%) had coronavirus; 4 (4.4%) had parainfluenza; and 6 (6.6%) had other viruses. Of those, 36 (39.6%) were diagnosed of ARDS; 50 (55.6%) were diagnosed of AHRF; and 49 (53.8%) were diagnosed of AKI.

Among the bacterial sepsis patients, 14/40 (35%) of culture proven cases were gram positive bacteria (most commonly Streptococcus, Staphylococcus, Enterococcus, and Gram-Positive Rod) and 22/40 (55%) were due to gram negative bacteria (most commonly Escherichia coli, Pseudomonas aeruginosa, Klebsiella pneumoniae, Acinetobacter baumannii, and gram-negative rod). Of those, 65 (34.4%) were diagnosed with ARDS; 71 (37.8%) were diagnosed with acute hypoxic respiratory failure (AHRF); and 114 (61.6%) were diagnosed with AKI.

### Measurement of Circulating Cytokines

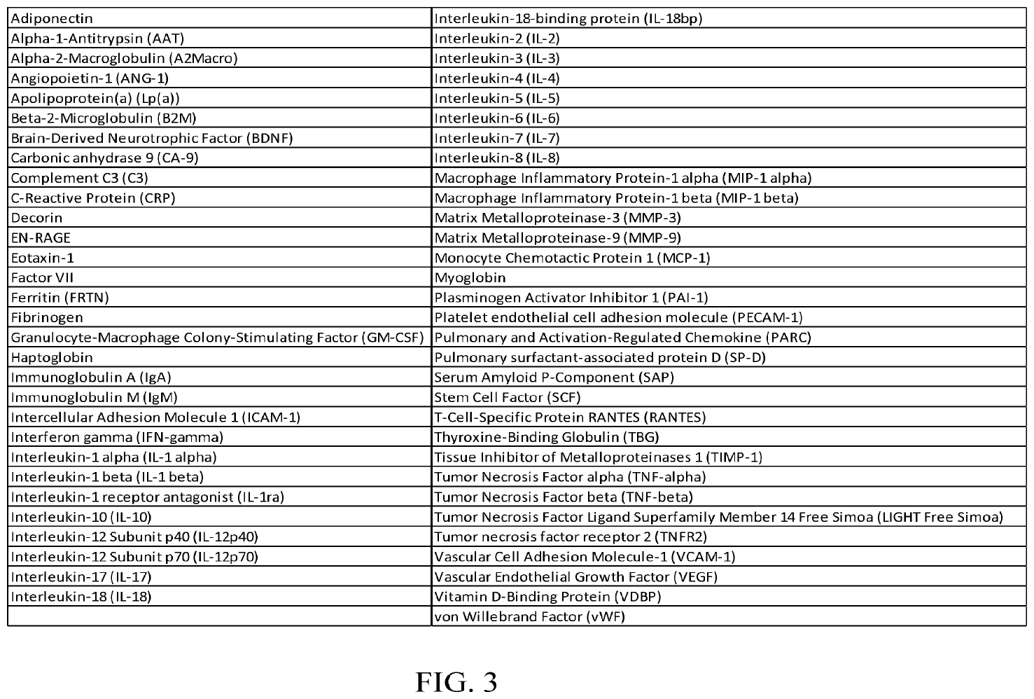

Plasma LIGHT, IL-18, and 59 other biomarkers (cytokines, chemokines and acute-phase reactants included in the Human Inflammation MAP® v. 1.1, plus extra 14 custom biomarkers) were measured by the Quanterix's single molecule array technology (Myriad RBM Simoa™ Services, Austin, TX). LIGHT and other biomarkers were also measured at the same time in 22 samples from random healthy subjects within comparable age, sex and ethnic background. The list of the biomarkers measured is shown in FIG. 3.

### Data Analysis

The plasma levels of the measured biomarkers were normalized by natural log transformation for further analysis. Elevated LIGHT level was defined as >2 standard deviations (SD) above mean in reference controls. Statistical analysis, including independent-samples t-test, One-Way ANOVA test, and bivariate correlations, was done by the IBM SPSS Statistics Version 23 software.

### Increased Plasma LIGHT Levels in Septicemia

Increased LIGHT levels were observed in the sepsis cohort of 280 patients overall, compared to control levels from random adult samples, and this was attributed to increased LIGHT levels in both the bacterial and viral subsets of sepsis (independent t-test, Table 2).

TABLE 2
Ln (LIGHT) and Ln (IL-18) levels in bacterial and viral sepsis

| | | Ln(LIGHT) | | Ln(IL-18) | |
|---|---|---|---|---|---|
| Group | n | x̅ ± s | P | x̅ ± s | P |
| Sepsis | 189 | 4.46 ± 0.71 | 1.80E−05 | 6.71 ± 0.91 | 0.038 |
| reference | 22 | 3.76 ± 0.64 | | 6.28 ± 0.87 | |
| controls | | | | | |
| Viral | 91 | 4.29 ± 0.72 | 1.78E−03 | 6.63 ± 0.90 | 0.048 |
| infection | | | | | |
| reference | 22 | 3.74 ± 0.70 | | 6.20 ± 0.89 | |
| controls | | | | | |

LIGHT levels were found to correlate with age (r=−0.120, p=0.045), i.e. older age correlated with lower levels of LIGHT, whereas sex, race and BMI did not show correlation with LIGHT levels. Patients with viral sepsis showed trend of having lower LIGHT levels than patients with bacterial sepsis, though not statistically different (P=0.060). In addition, as shown in FIG. 2, several other biomarkers demonstrated association with organ failure in the overall sepsis group, including both bacterial and viral sepsis.

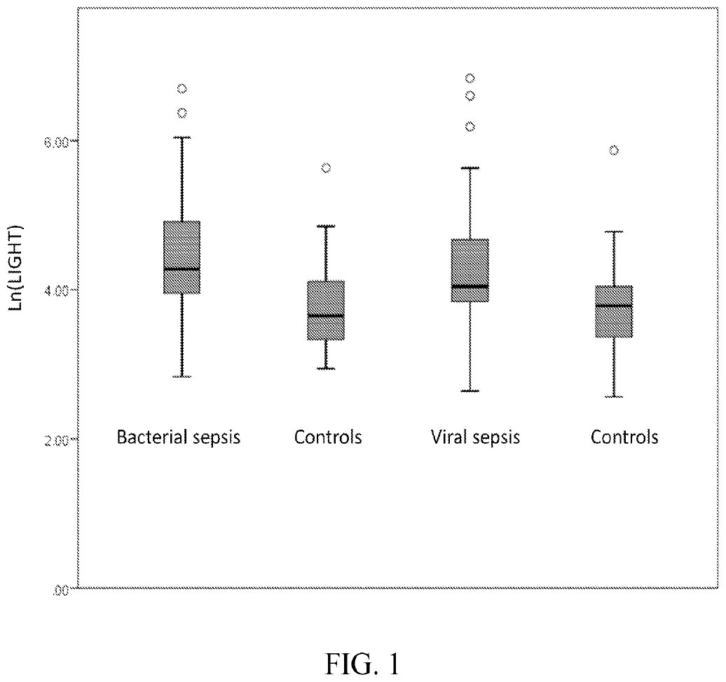

Overall, we observed increased LIGHT levels in both bacterial and viral sepsis patients (FIG. 1, Table 2). We defined abnormal LIGHT levels as higher than 2 times of standard deviation of the reference controls. In the 91 patients with viral sepsis, 12 (13.2%) cases had increased LIGHT. In the 189 bacterial sepsis cases, 40 (21.2%) cases had increased LIGHT, and 1/22 (4.5%) controls had elevated LIGHT. Overall, binary increased LIGHT was correlated with AHRF with r=0.123, P=0.043, and trend towards elevated Apache III score with r=0.114, P=0.060, also correcting with age.

In the bacterial sepsis cases, elevated LIGHT was correlated with Apache III, with r=0.172, p=0.020; associated with ARDS, with OR (95% CI)=2.180 (1.077, 4.414), p=0.028; associated with AHRF, with OR (95% CI)=2.037 (1.011, 4.106), p=0.044; and associated with AKI, with OR (95% CI)=2.179 (0.991, 4.791), p=0.049 (Table 3).

TABLE 3
Correlation of elevated LIGHT levels with organ failures^{#}

| | All cases | | Bacterial sepsis | | Viral sepsis | |
|---|---|---|---|---|---|---|
| LIGHT | r | p value | r | p value | r | p value |
| ARDS | 0.088 | 0.147 | 0.153* | 0.039 | −0.083 | 0.439 |
| AHRF | 0.123* | 0.043 | 0.144 | 0.052 | 0.073 | 0.498 |
| AKI | 0.084 | 0.169 | 0.159* | 0.031 | −0.106 | 0.322 |
| Apache | 0.114 | 0.060 | 0.172* | 0.02 | −0.027 | 0.802 |
| III | | | | | | |
| LOS | 0.014 | 0.823 | −0.056 | 0.453 | 0.207 | 0.052 |
| Mortality | 0.081 | 0.185 | 0.11 | 0.137 | −0.004 | 0.969 |

^{#}controlled for age.

In addition, we also observed inverse correlation between LIGHT concentrations and length of hospital stay (LOS) (r=−0.153, p=0.039).

These results suggest that LIGHT is a stronger pathogenic driver in bacterial sepsis than in viral sepsis, and given that both Apache III scores and mortality rates were higher in the bacterial sepsis lend support for elevated Apache III scores be considered an approach to identify patients that may have most benefit from LIGHT neutralizing mAb therapy.

### Variable Plasma IL-18 Levels in Septicemia

IL-18 levels were highly variable across individuals, ranging from 80 to >32,400 pg/mL in bacterial sepsis; and 161 to 19,100 pg/mL in viral sepsis. Compared to plasma LIGHT levels, the increase of plasma IL-18 levels showed only nominal significance in septicemia (Table 2). In our study, sex, age, and BMI did not show correlation with IL-18 levels. However, IL-18 levels were found to correlate with race, African Americans had lower IL-18 levels than other populations (6.44±0.98 vs. 6.79±0.85, p=0.003). In this case, instead of defining abnormal IL-18 levels, we tested correlation of quantitative IL-18 levels with clinical phenotypes, controlled for race (Table 4).

TABLE 4
Correlation of quantitative Ln(IL-18) levels with organ failures#

| | All cases | p | Bacterial sepsis | | Viral sepsis | |
|---|---|---|---|---|---|---|
| IL-18 | r | value | r | p value | r | p value |
| ARDS | 0.121* | 0.045 | 0.103 | 0.165 | 0.167 | 0.117 |
| AHRF | 0.145* | 0.017 | 0.101 | 0.175 | 0.251* | 0.018 |
| AKI | 0.187** | 0.002 | 0.151* | 0.041 | 0.257* | 0.015 |
| Apache | 0.301** | 0.000 | 0.278** | 1.40E−04 | 0.340** | 0.001 |
| III | | | | | | |
| LOS | 0.034 | 0.577 | 0.064 | 0.392 | −0.035 | 0.743 |
| Mortality | 0.288** | 0.000 | 0.254** | 0.001 | 0.350** | 0.001 |

#controlled for race

Overall, IL-18 levels were correlated with Apache III scores, mortality, and AKI, with highly significant p values; and correlated with AHRF and ARDS with nominal significance. In addition, the correlation of IL-18 with Apache III scores, mortality, and AKI, were consistently observed in both bacterial sepsis and viral sepsis. In contrast, the correlation of IL-18 with AHRF was only observed in viral sepsis. These results suggest that, unlike LIGHT with prominent pathological effects mainly observed in bacterial sepsis, IL-18 has significant pathological effects in both bacterial and viral sepsis.

### Biomarkers Correlated with Apache III Score in Both Bacterial and Viral Sepsis Cases

Among the 60 biomarkers, Tissue Inhibitor of Metalloproteinases 1 (TIMP-1), Tumor necrosis factor receptor 2 (TNFR2), Vascular Cell Adhesion Molecule-1 (VCAM-1), Plasminogen Activator Inhibitor 1 (PAI-1), IL-18, IL-18 Binding Protein (IL-18BP), myoglobin, Interleukin-6 (IL-6), von Willebrand Factor (vWF), Interleukin-8 (IL-8), ferritin (FRTN), Interleukin-1 receptor antagonist (IL-1Ra), Matrix Metalloproteinase-3 (MMP-3), Interleukin-10 (IL-10), Eotaxin-1, Macrophage Inflammatory Protein-1β (MIP-1β) and Interleukin-1β (IL-1β), were positively correlated with Apache III, in both bacterial and viral sepsis (FIG. 2).

Interestingly, complement C3, Factor VII, Vitamin D-Binding Protein (VDBP), Thyroxine-Binding Globulin (TBG), Serum Amyloid P-Component (SAP), Fibrinogen, and T-Cell-Specific Protein RANTES (RANTES), were all negatively correlated with Apache III score in both bacterial and viral sepsis (FIG. 2).

### Biomarkers Correlated with ARDS and AHRF in Both Bacterial and Viral Sepsis

In addition to the correlation of LIGHT as shown above, among the 60 biomarkers, PAI-1 was positively correlated with ARDS and AHRF, whereas fibrinogen was negatively correlated with ARDS (FIG. 2). Interestingly, IL-10, IL-6, IL-1Ra, IL-8, and TIMP-1, were correlated with ARDS and AHRF only in viral sepsis with highly statistical significance, which were not seen in bacterial sepsis.

### Biomarkers Correlated with AKI in Both Bacterial and Viral Sepsis

In addition to the association of LIGHT with AKI, multi-organ failures and worse clinical outcome measures, among the 60 additional biomarkers, B2M, Stem Cell Factor (SCF), TNFR2, VCAM-1, TIMP-1, Myoglobin, MMP-3, PAI-1, IL-18 and IL-18BP, were positively correlated with AKI, indicative of worse clinical outcome (FIG. 2).

### Biomarkers Correlated with Elevated LIGHT Levels

In viral sepsis, elevated LIGHT was positively correlated with RANTES. In bacterial sepsis, LIGHT was positively correlated with TIMP-1, TNFR2, PAI-1, IL-1Ra, vWF, IL-18BP, and B2M, while it was negatively correlated with Factor VII.

When comparing the damaging impact of LIGHT between the two groups of bacterial versus viral sepsis, ARDS and AHRF were found to be more common complications in patients with viral sepsis (consistent with the lungs being the primary infection site), while AKI is more commonly observed in patients with bacterial sepsis (FIG. 2).

### Biomarkers Correlated with IL-18 Levels

IL-18 levels correlated with disease biomarkers in both bacterial and viral sepsis. In both bacterial and viral sepsis, IL-18 levels were positively correlated with Ferritin, IL-10, IL-18BP, PAI-1, TIMP-1, TNFR2, VCAM-1, IL-8, MIP-1β, vWF, IL-1Ra, IL-6, B2M, and SCF; and negatively correlated with RANTES, Lp(a), and C3. In bacterial sepsis, IL-18levels were also positively correlated with Eotaxin-1 and MMP-3; and negatively correlated with Factor VII and Fibrinogen. In viral sepsis, IL-18 levels were also negatively correlated with SAP and VDBP. IL-18 levels weren't correlated with elevated LIGHT levels in either bacterial or viral sepsis (FIG. 2).

### Discussion

### LIGHT and IL-18 Levels as Biomarkers in Sepsis

We report elevated LIGHT levels in both bacterial and viral sepsis and association of LIGHT with ARDS and multi-organ failures in bacterial sepsis patients, including extended length of hospitalization, increased sepsis severity and sepsis complications. As shown here, both viral and bacterial sepsis cases were significantly correlated with increased levels of the pro-inflammatory cytokines IL-6, and IL-8³⁰, as well as with the anti-inflammatory cytokine IL-10, the acute phase protein IL-1Ra, and the matrix metalloproteinases (MMPs) inhibitor TIMP-1²⁹. However, these biomarkers were not correlated with LIGHT levels in either bacterial or viral sepsis.

IL-1Ra is an acute phase protein with potential anti-inflammatory effect by binding to IL-1 receptors³¹. Among these cytokines/biomarkers, the strongest correlation with ARDS/AHRF was from IL-10, suggesting ARDS/AHRF as a possible result of cytokine storm in viral infection³². These findings also emphasize inflammatory damage at the primary infection site in the lungs in non-COVID-19 viral infections.

IL-18 is a proinflammatory cytokine, inducing the production of interferon-γ (IFNγ)³³. Previous study has suggested IL-1 and IL-18 as potential therapeutic targets due to their crucial roles in sepsis⁵. In our study, while IL-1α and IL-1β were also measured, only IL-18 levels were consistently observed for correlations with increased sepsis severity and sepsis complications in both bacterial and viral sepsis. Highly significant correlations were observed between IL-18 and TIMP-1, the pro-inflammatory cytokines IL-10, IL-6, and IL-8, all of which were observed in both bacterial and viral sepsis. The potential of IL-18 as a precision therapeutic target in sepsis was also highlighted by its significantly individual variance and its extensive correlations with disease biomarkers. IL-18 levels weren't correlated with elevated LIGHT levels in either bacterial or viral sepsis, suggesting independent effects of LIGHT and IL-18 in sepsis rendering a combination therapy with neutralizing antibodies to LIGHT and IL 18 a therapeutically effective choice.

### Apache III Score Biomarkers

In bacterial sepsis, LIGHT is correlated with Apache III scores, AHRF and multi-organ failures, including ARDS and AKI, as well as length of hospital stay. The observed detrimental effects of LIGHT in organ failures are consistently observed to be associated with other biomarkers of organ failures. In bacterial sepsis, LIGHT is associated with other biomarkers of increased Apache III score, including TIMP-1, TNFR2, PAI-1, IL-1Ra, vWF, IL-18BP, and B2M. Unanimously, LIGHT is negatively correlated with biomarkers that are associated with decreased Apache III score, including Factor VII. Among these biomarkers, PAI-1 is also correlated with increased risk of ARDS and AHRF; TIMP-1, TNFR2, PAI-1, IL-18BP, and B2M are also correlated with increased risk of AKI.

The association of increased LIGHT level and Apache III score in bacterial sepsis observed in this study may be explained in part by its association with increased risk of ARDS, AHRF, and AKI, as discussed above. In addition to the correlation of other ARDS/AHRF and AKI biomarkers with LIGHT levels, LIGHT was also positively correlated with the Apache III score biomarkers, IL-1Ra and vWF in the bacterial sepsis cases. Previous study has shown that IL-1Ra is significantly associated with activation markers of coagulation, plasma levels of prothrombin fragment 1 and fragment 2 (F1+2), in severe infection³⁴. vWF plays a major role in blood coagulation by causing vWF-dependent platelet adhesion³⁵. Multivariate linear regression analysis of Apache III score shows independent effects of IL-10 (standardized coefficient Beta=0.199, p=0.005) and elevated LIGHT (standardized coefficient Beta=0.144, p=0.039), controlled for age, suggesting that LIGHT effects are independent of IL-10 mediated effects, such as cytokine storming in bacterial sepsis. This LIGHT effect was not seen in viral sepsis besides IL-10 effects. In addition, LIGHT is negatively correlated with various protective biomarker in sepsis, including Factor VII. Acquired factor VII deficiency is associated with severe systemic sepsis³⁶.

In both bacterial and viral sepsis, IL-18 is significantly correlated with Apache III score with high significance. In addition to the above biomarkers and related mechanisms shared by LIGHT, IL-18 is consistently correlated with TIMP-1, proinflammatory IL-10, IL-6, and IL-8. TIMP-1 is a key regulator of degradation of extracellular matrix. The matrix metalloproteinases (MMPs) degrade extracellular matrix and TIMP-1 is a natural inhibitor of MMPs³⁷. TIMP-1 also promotes cell proliferation and has anti-apoptotic function³⁸. In addition, IL-18 was consistently negatively correlated with protective biomarkers, RANTES, Lp(a), and C3, in both bacterial and viral sepsis. Among these biomarkers negatively associated with Apache III score, LIGHT was correlated with increased RANTES in viral sepsis. As shown previously, LIGHT is thought to induce the expression of RANTES through the lymphotoxin β receptor (LTβR) signaling³⁹. Unlike LIGHT, IL-18 was correlated with decreased RANTES levels in both bacterial and viral sepsis. Of note, RANTES is a chemokine with recognized pleiotropic activities, e.g. the beneficial effect of recruiting immune cells to areas of infection and the potential detrimental effects of enhancing the inflammatory processes⁴⁰. Lp(a) is the major protein component of HDL. HDL has anti-inflammatory properties and is diminished during inflammation⁴¹, which depletes Lp(a) in severe sepsis⁴². Complement depletion represented by decreased C3 also contributes to severe sepsis⁴³.

### Plasminogen Activator Inhibitor 1 (PAI-1) as Biomarker of ARDS and AHRF

PAI-1 levels show significant correlation with ARDS and AHRF are also positively correlated with LIGHT levels in bacterial sepsis; and positively correlated with IL-18 levels in both bacterial and viral sepsis. PAI-1 is the main physiological plasminogen activator inhibitor, which is critical for regulating the fibrinolytic system and maintaining normal hemostasis⁴⁴. Disseminated intravascular coagulation (DIC) is an important pathogenesis in early stage of acute lung injury (ALI) and ARDS⁴⁵. Due to the crucial role of the fibrinolytic system and DIC in the pathophysiology of sepsis, PAI-1 levels have been shown to be important prognostic biomarker in sepsis⁴⁶. Elevated in lung injury, alveolar PAI-1 levels can also predict ARDS in aspiration pneumonitis⁴⁷. Encoded by the serpin family E member 1 gene (SERPINE1), the expression of PAI-1 is upregulated by LIGHT⁴⁸. Therefore, the highly significant correction between LIGHT levels and PAI-1 levels identified by this study suggests that LIGHT may contribute to ARDS/AHRF in sepsis by increasing the PAI-1 levels.

### Biomarkers Associated with AKI

LIGHT levels in bacterial sepsis and IL-18 in both bacterial and viral sepsis were positively correlated with the ARDS/AHRF biomarkers, TIMP-1, TNFR2, PAI-1, IL-18BP, and B2M. B2M is a marker of proximal tubular injury in AKI⁴⁹. While PAI-1 suppresses fibrinolysis in DIC, TIMP-1 has also been suggested to play a role in the coagulation disturbance and disease severity of DIC⁵⁰. TNFR2 is the second receptor of the cytokines TNF and lymphotoxin-α, shown to mediate both proinflammatory and anti-inflammatory effects⁵¹. In recent years, TNFR2 has attracted research attention as an emerging drug target for autoimmune diseases and cancer⁵². In sepsis, TNFR2 has been shown to associate with CD4+ T-cell impairment and post-septic immunosuppression by activation of regulatory T cells (Treg)⁵³. Renal-expressed TNFR2 promotes renal monocyte recruitment by the IRF1 and IFN-β autocrine signaling, and may lead to renal injury⁵⁴. IL-18BP is the specific inhibitor of IL-18, and neutralizes IL-18 activities³³. Previous study has shown that IL-18 mediates ischemic acute tubular necrosis in AKI⁵⁵, whereas our study shows that IL-18BP has much stronger correlation with AKI than IL-18 in sepsis.

In conclusion, this study demonstrates independent associations of LIGHT and IL-18 in septic organ failures. Our study highlights two potential therapeutic targets in sepsis, i.e. the significantly increased LIGHT levels and the highly variable IL-18 levels across a subset of patients with septicemia. The detrimental roles of LIGHT in multi-organ failures were mainly seen in bacterial sepsis, including ARDS/AHRF, AKI, and its association with higher Apache III score as well as length of hospital stay with significant trend towards higher mortality rates. Consistently, the detrimental effects of LIGHT in bacterial sepsis were supported by the correlations of LIGHT with other biomarkers of organ failure suggesting a key role for LIGHT as an inflammatory driver of other detrimental mediators. Given that LIGHT presents an interesting therapeutic target for severe inflammatory conditions, our results suggest for the first time that anti-LIGHT therapy with neutralizing mAb may be effective in a subset of patients with septicemia unrelated to COVID-19 infections. In contrast, consistently detrimental roles of IL-18 were seen in both bacterial and viral sepsis, supported by the correlations of IL-18 with other biomarkers involved with organ failure. The observed significant variation in IL-18 levels in individuals with septicemia highlights its role as a precision therapeutic target with neutralizing mAb. The lack of correlation between LIGHT and IL-18 levels, as well as different correlations with other biomarkers, suggests independent and distinct roles of LIGHT and IL-18 in sepsis and that therapy directed against both of these cytokines could provide a therapeutic effect.

### REFERENCES

1. Croft M, Siegel R M. Beyond TNF: TNF superfamily cytokines as targets for the treatment of rheumatic diseases. Nature Reviews Rheumatology. 2017; 13(4):217.
2. Steinberg M W, Cheung T C, Ware C F. The signaling networks of the herpesvirus entry mediator (TNFRSF14) in immune regulation. Immunological reviews. 2011;244(1):169-187.
3. Ware C F, Šedý J R. TNF Superfamily Networks: bidirectional and interference pathways of the herpesvirus entry mediator (TNFSF14). Current opinion in immunology. 2011;23(5):627-631.
4. Wynn J L, Wilson C S, Hawiger J, et al. Targeting IL-17A attenuates neonatal sepsis mortality induced by IL-18. Proceedings of the National Academy of Sciences. 2016;113(19):E2627-E2635.
5. Berghe T V, Demon D, Bogaert P, et al. Simultaneous targeting of IL-1 and IL-18 is required for protection against inflammatory and septic shock. American journal of respiratory and critical care medicine. 2014; 189(3):282-291.
6. Remick D G. Pathophysiology of Sepsis. The American Journal of Pathology. 2007;170(5):1435-1444.
7. Mikkelsen M E, Shah C V, Meyer N J, et al. The epidemiology of acute respiratory distress syndrome in patients presenting to the emergency department with severe sepsis. Shock. 2013;40(5):375-381.
8. Bagshaw S M, George C, Bellomo R. Early acute kidney injury and sepsis: a multicentre evaluation. Critical care (London, England). 2008;12(2):R47.
9. Levi M, van der Poll T. Coagulation and sepsis. Thrombosis Research. 2017;149:38-44.
10. Strieter R, Lynch 3rd J, Basha M, Standiford T, Kasahara K, Kunkel S. Host responses in mediating sepsis and adult respiratory distress syndrome. Paper presented at: Seminars in respiratory infections1990.
11. Fry D E. Sepsis, systemic inflammatory response, and multiple organ dysfunction: the mystery continues. The American Surgeon. 2012;78(1):1-8.
12. Mauri D N, Ebner R, Montgomery R I, et al. LIGHT, a new member of the TNF superfamily, and lymphotoxin alpha are ligands for herpesvirus entry mediator. Immunity. 1998;8(1):21-30.
13. del Rio M L, Lucas C L, Buhler L, Rayat G, Rodriguez-Barbosa J I. HVEM/LIGHT/BTLA/CD160 cosignaling pathways as targets for immune regulation. Journal of leukocyte biology. 2010;87(2):223-235.
14. Cheung T C. Modulation of T cell proliferation through the LIGHT-HVEM-BTLA cosignaling pathway. Recent patents on DNA & gene sequences. 2009;3(3):177-182.
15. Celik S, Shankar V, Richter A, et al. Proinflammatory and prothrombotic effects on human vascular endothelial cells of Immune-cell-derived LIGHT. European Journal of Medical Research. 2009; 14(4):147.
16. Perlin D S, Zafir-Lavie I, Roadcap L, Raines S, Ware C F, Neil G A. Levels of the TNF-Related Cytokine LIGHT Increase in Hospitalized COVID-19 Patients with Cytokine Release Syndrome and ARDS. MSphere. 2020;5(4).
17. Arunachalam P S, Wimmers F, Mok C K P, et al. Systems biological assessment of immunity to mild versus severe COVID-19 infection in humans. Science. 2020;369(6508):1210-1220.
18. Haljasmägi L, Salumets A, Rumm A P, et al. Longitudinal proteomic profiling reveals increased early inflammation and sustained apoptosis proteins in severe COVID-19. Scientific reports. 2020;10(1):1-12.
19. Patel S, Saxena B, Mehta P. Recent updates in the clinical trials of therapeutic monoclonal antibodies targeting cytokine storm for the management of COVID-19. Heliyon. 2021:e06158.
20. Cheng T, Bai J, Chung C-S, Chen Y, Fallon E A, Ayala A. Herpes virus entry mediator (HVEM) expression promotes inflammation/organ injury in response to experimental indirect-acute lung injury. Shock (Augusta, Ga). 2019;51(4):487.
21. Baker K J, Houston A, Brint E. IL-1 Family Members in Cancer; Two Sides to Every Story. Front Immunol. 2019;10:1197.
22. Fabbi M, Carbotti G, Ferrini S. Context-dependent role of IL-18 in cancer biology and counter-regulation by IL-18BP. Journal of leukocyte biology. 2015;97(4):665-675.
23. Yu S, Chen Z, Mix E, et al. Neutralizing antibodies to IL-18 ameliorate experimental autoimmune neuritis by counter-regulation of autoreactive Th1 responses to peripheral myelin antigen. Journal of Neuropathology & Experimental Neurology. 2002;61(7):614-622.
24. Dinarello C, Novick D, Kim S, Kaplanski G. Interleukin-18 and IL-18 Binding Protein. Frontiers in Immunology. 2013;4(289).
25. Ranieri V M, Rubenfeld G D, Thompson B T, et al. Acute respiratory distress syndrome: the Berlin Definition. Jama. 2012;307(23):2526-2533.
26. Prescott H C, Sjoding M W, Langa K M, Iwashyna T J, McAuley D F. Late mortality after acute hypoxic respiratory failure. Thorax. 2018;73(7):618-625.
27. Kellum J A, Lameire N, Aspelin P, et al. Kidney disease: improving global outcomes (KDIGO) acute kidney injury work group. KDIGO clinical practice guideline for acute kidney injury. Kidney international supplements. 2012;2(1):1-138.
28. Knaus W A, Wagner D P, Draper E A, et al. The APACHE III prognostic system: risk prediction of hospital mortality for critically III hospitalized adults. Chest. 1991;100(6):1619-1636.
29. Visse R, Nagase H. Matrix metalloproteinases and tissue inhibitors of metalloproteinases: structure, function, and biochemistry. Circulation research. 2003;92(8):827-839.
30. Bester J, Pretorius E. Effects of IL-1B, IL-6 and IL-8 on erythrocytes, platelets and clot viscoelasticity. Scientific Reports. 2016;6(1):32188.
31. Arend W P, Malyak M, Guthridge C J, Gabay C. Interleukin-1 receptor antagonist: role in biology. Annual review of immunology. 1998;16:27-55.
32. Lu L, Zhang H, Dauphars D J, He Y-W. A Potential Role of Interleukin-10 in COVID-19 Pathogenesis. Trends in Immunology. 2020.
33. Dinarello C A. Interleukin-18 and the pathogenesis of inflammatory diseases. Seminars in nephrology. 2007;27(1):98-114.
34. Suharti C, van Gorp E C, Setiati T E, et al. The role of cytokines in activation of coagulation and fibrinolysis in dengue shock syndrome. Thrombosis and haemostasis. 2002;87(01):42-46.
35. Ruggeri Z M. von Willebrand factor. The Journal of clinical investigation. 1997;99(4):559-564.
36. Biron C, Bengler C, Gris J, Schved J. Acquired isolated factor VII deficiency during sepsis. Pathophysiology of Haemostasis and Thrombosis. 1997;27(2):51-56.
37. Wojtowicz-Praga S M, Dickson R B, Hawkins M J. Matrix metalloproteinase inhibitors. Investigational new drugs. 1997;15(1):61-75.
38. Ando T, Charindra D, Shrestha M, et al. Tissue inhibitor of metalloproteinase-1 promotes cell proliferation through YAP/TAZ activation in cancer. Oncogene. 2018;37(2):263-270.
39. Yeh DY-W, Wu C-C, Chin Y-P, Lu C-J, Wang Y-H, Chen M-C. Mechanisms of human lymphotoxin beta receptor activation on upregulation of CCL5/RANTES production. International Immunopharmacology. 2015;28(1):220-229.
40. Levy J A. The Unexpected Pleiotropic Activities of RANTES. The Journal of Immunology. 2009;182(7):3945-3946.
41. Ansell B J, Navab M, Watson K E, Fonarow G C, Fogelman A M. Anti-inflammatory properties of HDL. Reviews in Endocrine and Metabolic Disorders. 2004;5(4):351-358.
42. Van Leeuwen H J, Heezius E C, Dallinga G M, van Strijp J A, Verhoef J, van Kessel K P. Lipoprotein metabolism in patients with severe sepsis. Critical care medicine. 2003;31(5):1359-1366.
43. Ward P A. Role of the complement in experimental sepsis. Journal of leukocyte biology. 2008;83(3):467-470.
44. LIJNEN H R. Pleiotropic functions of plasminogen activator inhibitor-1. Journal of Thrombosis and Haemostasis. 2005;3(1):35-45.
45. Gando S, Kameue T, Matsuda N, Sawamura A, Hayakawa M, Kato H. Systemic inflammation and disseminated intravascular coagulation in early stage of ALI and ARDS: role of neutrophil and endothelial activation. Inflammation. 2004;28(4):237-244.
46. Iba T, Thachil J. Clinical significance of measuring plasminogen activator inhibitor-1 in sepsis. J Intensive Care. 2017;5:56-56.
47. El Solh A A, Bhora M, Pineda L, Aquilina A, Abbetessa L, Berbary E. Alveolar plasminogen activator inhibitor-1 predicts ARDS in aspiration pneumonitis. Intensive care medicine. 2006;32(1):110-115.
48. Otterdal K, Andreassen A K, Yndestad A, et al. Raised LIGHT levels in pulmonary arterial hypertension: potential role in thrombus formation. American journal of respiratory and critical care medicine. 2008;177(2):202-207.
49. Barton K T, Kakajiwala A, Dietzen D J, Goss C W, Gu H, Dharnidharka V R. Using the newer Kidney Disease: Improving Global Outcomes criteria, beta-2-microglobulin levels associate with severity of acute kidney injury. Clinical kidney journal. 2018;11(6):797-802.
50. Sivula M, Hästbacka J, Kuitunen A, et al. Systemic matrix metalloproteinase-8 and tissue inhibitor of metalloproteinases-1 levels in severe sepsis-associated coagulopathy. Acta anesthesiologica Scandinavica. 2015;59(2):176-184.
51. Medler J, Wajant H. Tumor necrosis factor receptor-2 (TNFR2): an overview of an emerging drug target. Expert opinion on therapeutic targets. 2019;23(4):295-307.
52. Medler J, Wajant H. Tumor necrosis factor receptor-2 (TNFR2): an overview of an emerging drug target. Expert Opin Ther Targets. 2019;23(4):295-307.
53. Gaborit B J, Roquilly A, Louvet C, et al. Regulatory T Cells Expressing Tumor Necrosis Factor Receptor Type 2 Play a Major Role in CD4+ T-Cell Impairment During Sepsis. The Journal of Infectious Diseases. 2020;222(7):1222-1234.
54. Venkatesh D, Ernandez T, Rosetti F, et al. Endothelial TNF receptor 2 induces IRF1 transcription factor-dependent interferon-β autocrine signaling to promote monocyte recruitment. Immunity. 2013;38(5):1025-1037.
55. Melnikov V Y, Faubel S, Siegmund B, Lucia M S, Ljubanovic D, Edelstein C L. Neutrophil-independent mechanisms of caspase-1- and IL-18-mediated ischemic acute tubular necrosis in mice. The Journal of clinical investigation. 2002;110(8):1083-1091.
56. Shui J W, Steinberg M W, Kronenberg M. Regulation of inflammation, autoimmunity, and infection immunity by HVEM-BTLA signaling. Journal of leukocyte biology. 2011;89(4):517-523.
57. Dolinay T, Kim Y S, Howrylak J, et al. Inflammasome-regulated cytokines are critical mediators of acute lung injury. Am J Respir Crit Care Med. 2012;185(11):1225-1234.
58. Singer M, Deutschman C S, Seymour C W, et al. The Third International Consensus Definitions for Sepsis and Septic Shock (Sepsis-3). Jama. 2016;315(8):801-810.
59. Vasarmidi E, Tsitoura E, Spandidos D A, Tzanakis N, Antoniou K M. Pulmonary fibrosis in the aftermath of the COVID-19 era. Experimental and therapeutic medicine. 2020;20(3):2557-2560.
60. Reilly J P, Anderson B J, Hudock K M, et al. Neutropenic sepsis is associated with distinct clinical and biological characteristics: a cohort study of severe sepsis. Critical care (London, England). 2016;20(1):222.
61. Reilly J P, Wang F, Jones T K, et al. Plasma angiopoietin-2 as a potential causal marker in sepsis-associated ARDS development: evidence from Mendelian randomization and mediation analysis. Intensive care medicine. 2018;44(11):1849-1858.
62. Steinhauser M L, Hogaboam C M, Kunkel S L, Lukacs N W, Strieter R M, Standiford T J. IL-10 is a major mediator of sepsis-induced impairment in lung antibacterial host defense. The Journal of Immunology. 1999;162(1):392-399.

1. A method of treating sepsis in patients having an elevated APACHE III score 2 standard deviations above the mean observed in control subjects or an elevated APACHE III score in the range of 200-299 indicating a need for treatment, the method comprising administering to the patient an effective amount of at least one sepsis biomarker inhibitor.

2. (canceled)

3. A method of treating sepsis in a patient in need thereof, comprising:
a. determining whether the patient harbors an elevated level of at least one sepsis biomarker, and
b. administering an effective amount of at least one sepsis biomarker inhibitor.

4. The method of claim 3, wherein the at least one sepsis biomarker includes at least one of
i) LIGHT and the sepsis biomarker inhibitor is a LIGHT antagonist or an anti-LIGHT antibody;
ii) IL-18 and said at least one inhibitor inhibits IL-18 activity:
iii) TIMP-1 and said at least one inhibitor inhibits TIMP-1 activity;
iv) TNFR2 and said at least one inhibitor inhibits TNFR2 activity;
v) VCAM-1 and said at least one inhibitor inhibits VCAM-1 activity:
vi) PAI-1 and said at least one inhibitor inhibits PAI-1 activity;
vii) IL-18BP and said at least one inhibitor inhibits IL-18BP activity;
viii) IL-6 and said at least one inhibitor inhibits IL-6 activity;
ix) vWF and said at least one inhibitor inhibits vWF activity;
x) IL-8 and said at least one inhibitor inhibits IL-8 activity;
xi) FRTN and said at least one inhibitor inhibits FRTN activity;
xii) IL-1Ra and said at least one inhibitor inhibits IL-1Ra activity;
xiii) MMP-3 and said at least one inhibitor inhibits MMP-3 activity;
xiv) IL-10 and said at least one inhibitor inhibits IL-10 activity;
xv) Eotaxin-1 and said at least one inhibitor inhibits Eotaxin-1 activity;
xvi) MIP-1B and said at least one inhibitor inhibits MIP-1B activity; and
xvii) includes IL-1β and said at least one inhibitor inhibits IL-1β activity.

5. (canceled)

6. The method of claim 3, wherein the at least one sepsis marker includes
i) IL-10 and said at least one inhibitor inhibits IL-10 activity;
ii) IL-6 and said at least one inhibitor inhibits IL-6 activity;
iii) IL-1Ra and said at least one inhibitor inhibits IL-1Ra activity;
iv) IL-8 and said at least one inhibitor inhibits IL-8 activity;
v) TIMP-1 and said at least one inhibitor inhibits TIMP-1 activity; and
iv) PAI-1 and said at least one inhibitor inhibits PAI-1 activity.

7. The method of claim 3, wherein the at least one sepsis marker includes
i) B2M and said at least one inhibitor inhibits B2M activity;
ii) Stem Cell Factor (SCF) and said at least one inhibitor inhibits SCF activity;
iii) TNFR2 and said at least one inhibitor inhibits TNFR2 activity;
iv) VCAM-1 and said at least one inhibitor inhibits VCAM-1 activity;
v) TIMP-1 and said at least one inhibitor inhibits TIMP-1 activity;
vi) myoglobin and said at least one inhibitor inhibits myoglobin;
vii) MMP-3 and said at least one inhibitor inhibits MMP-3;
vii) PAI-1 and said at least one inhibitor inhibits PAI-1;
viii) IL-18 and said at least one inhibitor inhibits IL-18; and
ix) IL18BP and said at least one inhibitor inhibits IL18BP.

8-20. (canceled)

21. The method of claim 3, wherein the at least one sepsis marker includes complement C3 and said at least one inhibitor inhibits complement C3 activity.

22. The method of claim 3, wherein the at least one sepsis marker includes Factor VII and said at least one inhibitor inhibits Factor VII activity.

23. The method of claim 3, wherein the at least one sepsis marker includes Vitamin D-Binding Protein (VDBP) and said at least one inhibitor inhibits VDBP activity.

24. The method of claim 3, wherein the at least one sepsis marker includes Thyroxine-Binding Globulin (TBG) and said at least one inhibitor inhibits TBG activity.

25. The method of claim 3, wherein the at least one sepsis marker includes Serum Amyloid P-Component (SAP) and said at least one inhibitor inhibits SAP activity.

26. The method of claim 3, wherein the at least one sepsis marker includes Fibrinogen and said at least one inhibitor inhibits Fibrinogen activity.

27. The method of claim 3, wherein the at least one sepsis marker includes T-Cell-Specific Protein RANTES (RANTES) and said at least one inhibitor inhibits RANTES activity.

28. The method of claim 3, wherein step a further comprises:
a. determining whether the patient harbors:
i. an elevated level of at least two sepsis biomarkers selected from LIGHT, TIMP-1, TNFR2, VCAM-1, PAI-1, IL-18, IL-18BP, IL-6, vWF, IL-8, FRTN, IL-1Ra, MMP-3, IL-10, Eotaxin-1, MIP-1B, and IL-1β when compared to the mean observed in control subjects; and/or
ii. a lower level of at least two sepsis biomarkers selected from complement C3, Factor VII, Vitamin D-Binding Protein (VDBP), Thyroxine-Binding Globulin (TBG), Serum Amyloid P-Component (SAP), Fibrinogen, and T-Cell-Specific Protein RANTES (RANTES) when compared to the mean observed in control subjects.

29. A method of treating Acute Respiratory Distress Syndrome (ARDS) in a patient in need thereof, comprising:
a. determining whether the patient harbors:
i. an elevated of at least two sepsis biomarkers selected from IL-10, IL-6, IL-1Ra, IL-8, TIMP-1, and PAI-1 when compared to the mean observed in control subjects; and/or
ii. a lower level fibronectin when compared to the mean observed in control subjects; and
b. administering an effective amount of at least one sepsis biomarker inhibitor.

30. The method of claim 3 wherein the patient has Acute Kidney Injury (AKI) and step a further comprises
determining whether the patient harbors an elevated of at least two sepsis biomarker selected from B2M, Stem Cell Factor (SCF), TNFR2, VCAM-1, TIMP-1, Myoglobin, MMP-3, PAI-1, IL-18, and IL18BP.
